(19) United States
(12) Patent Application Publication
(10) Pub. No.: US 2008/0177137 A1
(43) Pub. Date: Jul. 24, 2008

(54) ELECTRONIC ENDOSCOPE APPARATUS

(75) Inventor: Akira MATSUI, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: Olympus Corporation, Tokyo (JP)

(21) Appl. No.: 11/844,063

(22) Filed: Aug. 23, 2007

(30) Foreign Application Priority Data
Sep. 15, 2006 (JP) 2006-251512

(51) Int. Cl. A61B 1/04

(52) U.S. Cl. 600/109

(57) ABSTRACT

An electronic endoscope apparatus has an endoscope unit and an image processing unit. The endoscope unit is configured to be inserted into the subject's body cavity and has a first communication control unit configured to perform bidirectional communication with the image processing unit via a signal line in time-division fashion. The image processing unit has a second communication control unit configured to perform bidirectional communication with the endoscope unit via the signal line in time-division fashion. The second communication control unit transmits a signal to the endoscope unit during a period in which no signals are transmitted to it from the first communication control unit.

### CROSS-REFERENCE TO RELATED APPLICATIONS

This application is based upon and claims the benefit of priority from prior Japanese Patent Application No. 2006-251512, filed Sep. 15, 2006, the entire contents of which are incorporated herein by reference.

### BACKGROUND OF THE INVENTION

1. Field of the Invention

The present invention relates to an electronic endoscope apparatus that comprises an imaging element provided in the distal part and an optical control unit provided in the distal part, too, and configured to focus an optical image on the imaging element.

2. Description of the Related Art

Endoscope apparatuses are widely used, each enabling a doctor to examine the organs and the like present in a body cavity, while the thin insertion section remains inserted into the body cavity. It is demanded that the insertion section of the endoscope apparatus be thinner so that it may be easily inserted into body cavities and the distress of the patient may be reduced.

In recent years, electronic endoscope apparatuses have come into use, each having, in the distal part, an imaging element such as a CCD that converts optical images into image signals. In any electronic endoscope apparatuses, an image signal representing an image focused on the imaging element is transmitted from the endoscope unit to a processor unit that is an external apparatus. The processor unit produces a video signal. The video signal is supplied to a monitor connected to the processor unit. The monitor displays the endoscopic image represented by the video signal. Seeing the endoscopic image, the doctor examines the organ or the like.

The processor unit not only receives image signals from the imaging element, but also outputs various drive signals for driving the imaging element. Therefore, a plurality of signal lines, such as image-signal reading lines and drive-signal lines for the imaging element, extend through the insertion section of the endoscope unit of the electronic endoscope apparatus. This inevitably prevents the insertion section from becoming thinner as desired.

Jpn. Pat. Appln. KOKAI Publication No. 11-32982, for example, discloses an electronic endoscope apparatus. In this apparatus, the imaging element, and the drive signal generating unit, the signal-processing unit and the like, which are conventionally provided in the processor unit, are all integrated in one chip and are provided in the distal part of the endoscope unit. Thus, less signal lines extend between the endoscope unit and the processor unit. As a result, the insertion section of the endoscope unit is thinner than in the conventional electronic endoscope apparatuses.

### BRIEF SUMMARY OF THE INVENTION

According to a first aspect of the present invention, there is provided an electronic endoscope apparatus comprising: an endoscope unit which has a first communication control unit configured to be inserted into a body cavity of a subject and to perform bidirectional communication via a signal line in time-division fashion; and an image processing unit which has a second communication unit configured to perform bidirectional communication with the endoscope unit via the signal line in time-division fashion and to transmit a signal from the image processing unit to the endoscope unit during a period in which no signals are transmitted from the first communication control unit.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing the overall configuration of an electronic endoscope system having an electronic endoscope apparatus according to an embodiment of this invention;

FIG. 2 is a block diagram showing the major components of the endoscope unit 1 and the processor unit 2, both provided in the electronic endoscope system;

FIG. 3 is a block diagram illustrating the internal structures of transmitting/receiving circuits 19 and 21;

FIG. 4 is a timing chart representing the relation between an output-enable signal A, an output-enable signal B and an image signal;

FIG. 5 is a chart explaining bidirectional communication in which a image signal and a control signal are time-divided; and

FIG. 6 is a diagram showing the configuration of a control signal.

### DETAILED DESCRIPTION OF THE INVENTION

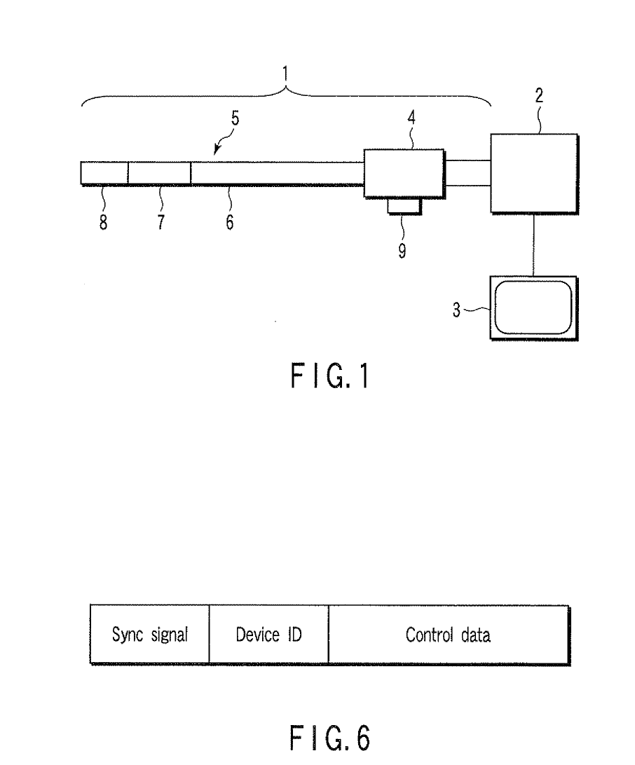

FIG. 1 is a schematic diagram showing the overall configuration of an electronic endoscope system having an electronic endoscope apparatus according to an embodiment of the present invention.

As FIG. 1 shows, the electronic endoscope system is composed of an electronic endoscope apparatus and a monitor unit 3. The electronic endoscope apparatus comprises an endoscope unit 1 and a processor unit 2. The monitor unit 3 is connected to the electronic endoscope apparatus. The endoscope unit 1 incorporates an imaging element such as a CCD. The unit 1 photographs the interior of the subject's body and generates an image signal representing the image of the interior of the body. The processor unit 2 processes the image signal, generating a video signal. The video signal is supplied to the monitor unit 3. The monitor unit 3 displays the image represented by the video signal.

The electronic endoscope system shown in FIG. 1 will be further described. As shown in FIG. 1, the endoscope unit 1 has a long thin insertion section 5 and an operation section 4. The insertion section 5 is inserted into a body cavity in which an object of observation exists. The operation section 4 is connected to the proximal end of the insertion section 5. The insertion section 5 has a rigid distal part 8, a bending part 7, and a soft flexible part 6. The rigid distal part 8 contains an imaging element. The flexible part 6 is connected to the bending part 7. The bending part 7 is connected to the rear end of the distal part B and can be bent as the operation knob (not shown) provided on the operation section 4 is operated.

A switch pad 9 is provided on the operation section 4 of the endoscope unit 1. As the switch pad 9 is operated, the processor unit 2 operates to control the LED and the zoom mechanism, both incorporated in the distal part 8. There amount of illumination light and the image magnification can therefore be changed.

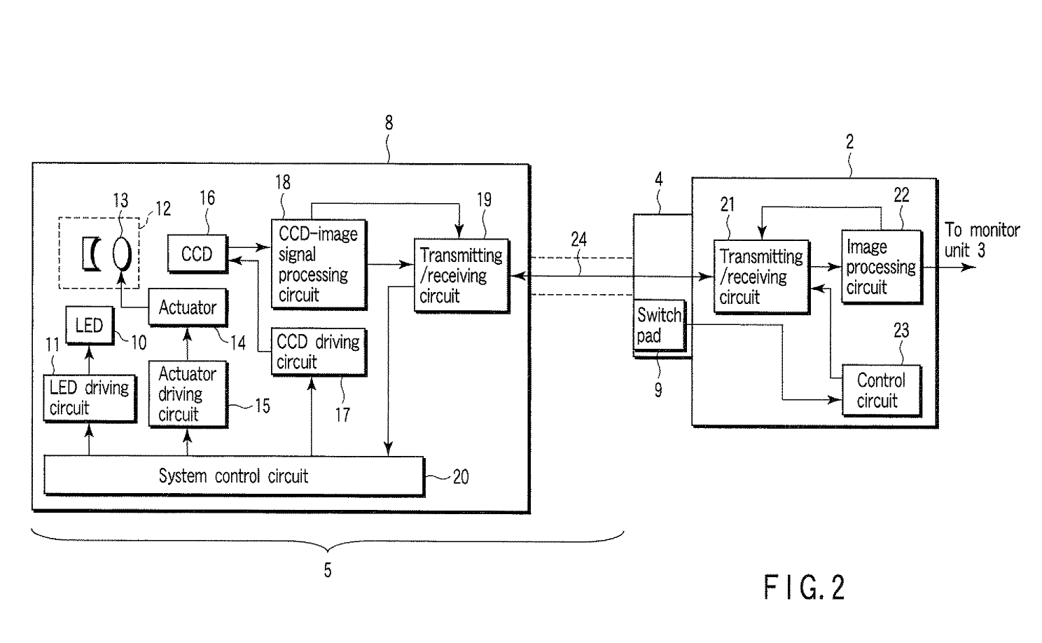

FIG. 2 is a block diagram showing the major components of the endoscope unit 1 and processor unit 2 of the electronic endoscope system. The distal part 8 of the endoscope unit 1 incorporates an LED 10, an LED driving circuit 11, an objective optical system 12, an actuator 14, an actuator driving circuit 15, a CCD 16, a CCD driving circuit 17, a CCD-image signal processing circuit 18, a transmitting/receiving circuit 19, and a system control circuit 20.

The LED 10 is a light source that illuminates the photographing region in a body cavity, enabling the CCD 16 to photograph the object of observation. The LED driving circuit 11 turns on and off the LED 10. The circuit 11 also controls the LED 10, changing the amount of light the LED 10 emits. The objective optical system 12 is an optical system designed to focus the light reflected from the photographing region illuminated by the LED 10, thus forming an image on the CCD 16. The objective optical system 12 has a zoom lens 13, which functions an optical control unit. The actuator 14 can move the zoom lens 13 back and forth along the optical axis thereof. As the zoom lens 13 is so moved, the image observed can be changed in magnification. The actuator 14 is driven by a drive signal supplied from the actuator driving circuit 15. The actuator 14 may be, for example, a piezoelectric actuator, an ultrasonic actuator or a motor.

The CCD 16, which functions as an imaging unit, is an imaging element of CCD type that performs photoelectric conversion on the light reflected from the photographing region. The CCD driving circuit 17 drives the CCD 16, controlling the operation thereof. The CCD-image signal processing circuit 18 processes any analog signal output from the CCD 16, generating an image signal.

The transmitting/receiving circuit 19, which is the first communication control unit, transmits any image signal output from the CCD-image signal processing circuit 18, to the processor unit 2. The circuit 19 also receives control signals output from the processor unit 2.

The system control circuit 20, which functions as a control unit, controls the LED driving circuit 11, actuator driving circuit 15 and CCD driving circuit 17 in accordance with the control signals supplied from the processor unit 2 through the transmitting/receiving circuit 19.

The processor unit 2 comprises a transmitting/receiving circuit 21, an image processing circuit 22, and a control circuit 23. The transmitting/receiving circuit 21, which is the second communication control unit, receives an image signal transmitted from the distal part 8 via a signal line 24 that extends through insertion section 5 of the endoscope unit 1. The transmitting/receiving circuit 21 also transmits the control signals output from the control circuit 23, to the distal part 8 of the endoscope unit 1 through the signal line 24. The control signals are signals that will control the LED driving circuit 11, actuator driving circuit 15 and CCD driving circuit 17, respectively. The image processing circuit 22 processes an image signal it has received via the transmitting/receiving circuit 21, thus generating a video signal. The video signal is supplied to the monitor unit 3, which displays the image to be observed. The control circuit 23 detects the operated state of the switch pad 9 provided on the operation section 4 of the endoscope unit 1 that is connected the processor unit 2. On the basis of the operated state, the control circuit 23 generates control signals for controlling the LED 10, actuator 14 and the like that are provided in the distal part 8. When the switch pad 9 is operated to change, for example, the amount of light the LED 10 emits, the control circuit 23 produces a control signal. This control signal is output to the system control circuit 20 provided in the distal part 8. When the switch pad 9 is operated to change the image magnification, or to adjust the focusing at the objective optical system 12, the control circuit 23 produces a control signal for changing the magnification or for adjusting the focusing. The control signal is output to the system control circuit 20.

How signals are exchanged between the endoscope unit 1 and the processor unit 2 will be explained. In the electronic endoscope system (FIG. 1), an image signal generated in the CCD 16 is transmitted from the endoscope unit 1 to the processor unit 2. And control signals are transmitted from the processor unit 2 to the endoscope unit 1 to control the LED driving circuit 11, actuator driving circuit 15 and CCD driving circuit 17 that are provided in the distal part 8 of the endoscope unit 1.

First, the CCD-image signal processing circuit 18 samples, in unit of pixels, an analog signal output from the CCD 16, which represents the luminance values of pixels forming the image focused on the CCD 16. The analog signal thus sampled is thereby converted to digital data of, for example, 8-bit gradation. This digital image signal is subjected to modulation such as 8B10B. Sync signals are inserted into the image signal thus modulated, so that the image data may be synchronized in the processor unit 2 that receives the image signal. More specifically, horizontal sync signals are inserted at the heads of the lines constituting one frame, each indicating the head of one line, and vertical sync signals are inserted at the heads of one-frame data items, each indicating the head of one frame. The sync signals are bit patterns, which will never appear as data items representing pixels. Hence, the image signal can be distinguished from the sync signals in the processor unit 2 that receives the image signal and sync signals.

The image signal, now containing horizontal sync signals and vertical sync signals, is output to the transmitting/receiving circuit 19 as parallel data. At this point, the CCD-image signal processing circuit 18 generates an output-enable signal A in synchronism with one frame of the image signal. The output-enable signal A and the image signal are output to the transmitting/receiving circuit 19. The output-enable signal A is a signal that is high during the image-signal transfer period and low during the other period (blanking period).

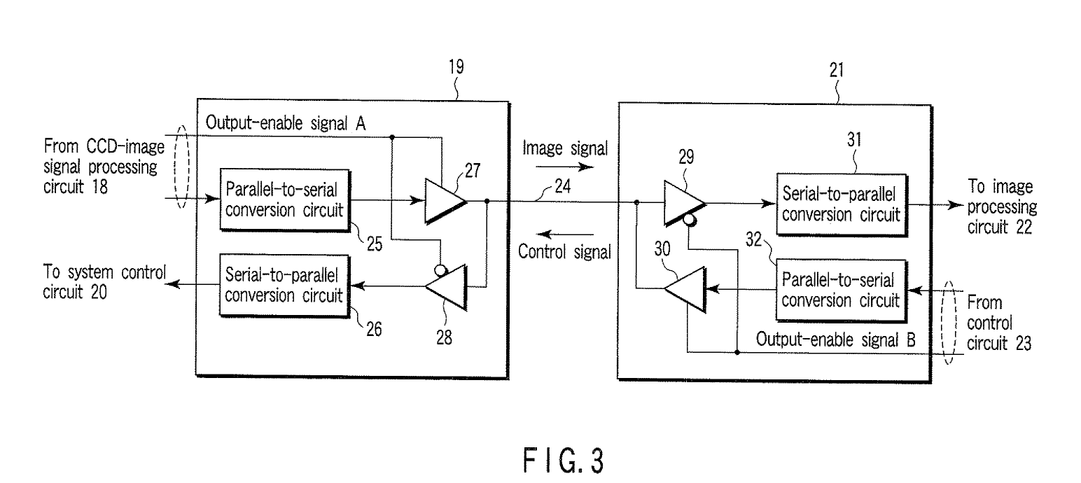

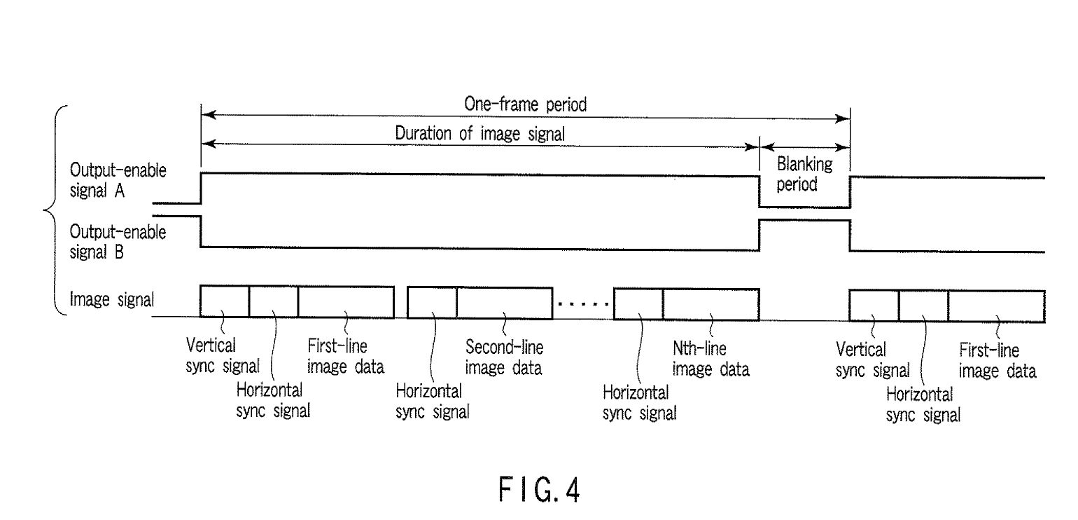

FIG. 3 is a block diagram illustrating the internal structures of transmitting/receiving circuits 19 and 21. In the transmitting/receiving circuit 19, a parallel-to-serial conversion circuit 25 converts the input data to serial data. The serial data is output to the signal line 24 through a transmission buffer 27. Then, as shown in FIG. 4, the output-enable signal A is controlled to be high for the duration of one frame of the image signal. While the output-enable signal A is high, the output of the transmission buffer 27 is valid. The serial data representing the image signal is therefore output to the signal line 24. On the other hand, the output of a reception buffer 28 is invalid. No data is therefore output to a serial-to-parallel conversion circuit 26.

In the processor unit 2, the output-enable signal B of the transmitting/receiving circuit 21 is controlled to be low level for the duration of the image signal. While the output-enable signal B is low, no output of a transmission buffer 30 is supplied to the signal line 24, and the image signal output from the transmission buffer 27 of the transmitting/receiving circuit 19 is supplied to a reception buffer 29. The image signal output from the reception buffer 29 is supplied to a serial-to-parallel conversion circuit 31. The circuit 31 converts the image signal to parallel data. The parallel data is output from the transmitting/receiving circuit 21 to the image processing circuit 22. From the parallel data the image processing circuit 22 generates a video signal. The video signal is supplied to the monitor unit 3. The monitor unit 3 displays the image photographed by the endoscope unit 1.

How control signals are transmitted from the processor unit 2 to the endoscope unit 1 will be explained. Assume that the switch pad 9 is operated to accomplish zooming, thereby to magnify the image, for example, three times. The signal the switch pad 9 has produced is transmitted through the operation section 4 to the control circuit 23 of the processor unit 2. The control circuit 23 generates a control signal corresponding to the signal produced by the switch pad 9. The control signal is output to the transmitting/receiving circuit 21. In this case, the control circuit 23 generates a control signal that will drive the actuator 14 to move the zoom lens 13 to the three-times zoom position.

The control signal generated by the control circuit 23 is transmitted from the transmitting/receiving circuit 21 to the transmitting/receiving circuit 19 through the same signal line 24 that has conveyed the image signal from the endoscope unit 1 to the processor unit 2. That is, the control signal is transmitted from the circuit 21 to the circuit 19 during the vertical blanking period of the image signal. As described above, the transmission buffer 27 is controlled so that its output may be valid only for the duration of the image signal to transmit. The output of the transmission buffer 27 is therefore invalid during the vertical blanking period of the image signal.

In the processor unit 2, the image processing circuit 22 detects vertical sync signals and horizontal sync signals from the image signal received via the transmitting/receiving circuit 21. Thus, the image processing circuit 22 reproduces frame data in synchronism with these sync signals. The control circuit 23 generates an output-enable signal B in synchronism with the image signal received. The output-enable signal B is supplied to the transmitting/receiving circuit 21. As shown in FIG. 4, the output-enable signal B is high during the vertical blanking period and low during all other periods. While the output-enable signal B is high, the output of the transmission buffer 30 of the transmitting/receiving circuit 21 is valid. Hence, the control signal can be transmitted from the transmission buffer 30 of the transmitting/receiving circuit 21 to the reception buffer 28 of the transmitting/receiving circuit 19 through the signal line 24.

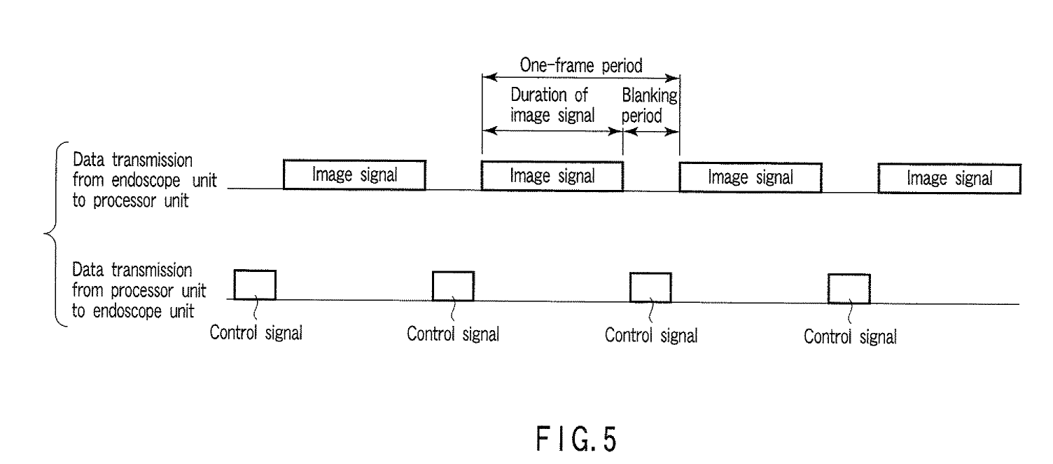

As a result, signals are exchanged between the endoscope unit 1 and the processor unit 2 in time-division fashion at such timing as is illustrated in FIG. 5, by utilizing the signal line 24,.

A control signal to be transmitted from the processor unit 2 to the endoscope unit 1 will be described. The control signal has such a data configuration as shown in FIG. 6. That is, as FIG. 6 shows, the control signal is composed of a sync-signal pattern, a device ID, and control data, as shown in FIG. 6. The sync-signal pattern is a pattern of sync signals used for synchronizing the control signal received by the transmitting/receiving circuit 19 in the endoscope unit 1. The device ID is ID information designating that component of the endoscope unit 1 which should be controlled by the system control circuit 20. In the case described above, the device ID designates the actuator driving circuit 15. The control data is data that the system control circuit 20 should control uses to control the device that should be controlled in the endoscope unit. In the case described above, the control data makes the actuator driving circuit 15 drives the actuator 14 to perform three-times zooming.

The control signal of the above-described data configuration is transmitted from the transmitting/receiving circuit 21 to the transmitting/receiving circuit 19. The control signal is then converted to parallel data by the serial-to-parallel conversion circuit 26. The parallel data is supplied to the system control circuit 20. The system control circuit 20 controls a drive circuit to drive that component of the endoscope unit 1, which the device ID designates. In the case specified above, the circuit 20 controls the actuator driving circuit 15, whereby the actuator 14 is driven to move the zoom lens 13 to the three-times zoom position.

As described above, in the present embodiment, one signal line 24 connects the transmitting/receiving circuit 19 provided in the distal part 8 of the endoscope unit 1 to the transmitting/receiving circuit 21 provided in the processor unit 2. Through this signal line 24, control signals are transmitted from the processor unit 2 to the endoscope unit 1 to control the actuator 15 and the like, during the vertical blanking period of the image signal. Thus, both the image signal and any control signal can be transmitted through the signal line 24 that connects the distal part 8 and the processor unit 2, in time-division fashion by virtue of the bidirectional communication between the endoscope unit 1 and the processor unit 2. As a result, no other signal lines need be provided to transmit control signals even if mechanisms, such as a zoom mechanism, focusing mechanism and a variable diaphragm, are added in the distal part 8 of the endoscope unit 1. That is, signal lines other than the signal line 24 need not be added to transmit control signals. This helps to maintain the small diameter of the insertion section 5 of the endoscope unit 1.

As has been pointed out, any control signal transmitted from the processor unit 2 to the endoscope unit 1 contains a device ID that designates that component provided in the distal part 8 of the endoscope unit 1, which should be controlled by the control signal. The system control circuit 20 controls a drive circuit to drive the component the device ID designates. Hence, more signal lines need not be provided in the insertion section 5 of the endoscope unit I even if the distal part 8 of the endoscope unit 1 incorporates more devices that should be controlled. The small diameter of the insertion section 5 of the endoscope unit 1 can therefore be maintained.

In the embodiment described above, the distal part 8 of the endoscope unit 1 incorporates a zoom mechanism of the objective optical system 12, which should be controlled by a control signal. Nonetheless, the distal part 8 may incorporate other optical control components that should be controlled, such as a focusing mechanism and a variable diaphragm mechanism.

In the embodiment described above, the control signals are transmitted from the processor unit 2 to the endoscope unit 1 during the vertical blanking period of the image signal. Instead, the control signals may be transmitted to the endoscope unit 1 during the horizontal blanking period of the image signal (i.e., the spaces between the segments of the image signal, shown in FIG. 4). Furthermore, signals other than control signals may be exchanged between the endoscope unit 1 and the processor unit 2 during the vertical blanking period of the image signal. For example, a position signal generated by an encoder, if any provided in the distal part 8 to detect the position of the zoom lens 13, may be transmitted from the endoscope unit 1 to the processor 2. In this case, a feedback control can be achieved, using the position signal, and any control signal produced in the processor unit 2 may be transmitted to the endoscope unit 1 during that part of the vertical blanking period, during which neither the position signal nor the control signals are transmitted.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An electronic endoscope apparatus comprising:
an endoscope unit which has a first communication control unit configured to be inserted into a body cavity of a subject and to perform bidirectional communication via a signal line in time-division fashion; and
an image processing unit which has a second communication unit configured to perform bidirectional communication with the endoscope unit via the signal line in time-division fashion and to transmit a signal from the image processing unit to the endoscope unit during a period in which no signals are transmitted from the first communication control unit.

2. The electronic endoscope apparatus according to claim 1, wherein a signal transmitted from the first communication control unit contains an image signal, and the period in which no signals are transmitted from the first communication control unit is a blanking period of the image signal.

3. The electronic endoscope apparatus according to claim 2, wherein the first communication control unit controls the bidirectional communication in accordance with vertical sync signals contained in the image signal, and the second communication control unit controls the bidirectional communication in accordance with vertical sync signals reproduced from the image signal.

4. The electronic endoscope apparatus according to claim 1, wherein the endoscope unit includes an imaging unit which photographs the interior of the body cavity and generates the image signal, at least one optical control unit which forms, at the photographing unit, an optical image of the interior of the body cavity, and a control unit which controls the optical control unit; and a control signal is transmitted from the second communication control unit to the control unit to control the optical control unit.

5. The electronic endoscope apparatus according to claim 4, wherein the control signal contains ID data for identifying the optical control unit that should be controlled by the control unit, and the control unit controls the optical control unit identified by the ID data.
